(19) United States
(12) Patent Application Publication
(10) Pub. No.: US 2009/0247831 A1
(43) Pub. Date: Oct. 1, 2009

(54) ENDOSCOPE, DISTAL END CAP-EQUIPPED ENDOSCOPE AND ENDOSCOPE CLEANING SHEATH

(76) Inventors: Shinichi MIYAMOTO, Hachioji-shi (JP); Akira SUZUKI, Uenohara-shi (JP)

Correspondence Address: OSTROLENK FABER GERB & SOFFEN, 1180 AVENUE OF THE AMERICAS, NEW YORK, NY, 100368403, US

(21) Appl. No.: 12/410,734

(22) Filed: Mar. 25, 2009

(30) Foreign Application Priority Data
Mar. 31, 2008 (JP) 2008-091918

(51) Int. Cl. A61B 1/12

(52) U.S. Cl. 600/157

(57) ABSTRACT

An endoscope includes, an insertion section, a distal end section body which constitutes a distal end section of the insertion section and has at least an observation window, a liquid feed path which is formed to supply a liquid to the distal end section body side and communicates with a liquid feed source, a gas feed path which is formed to supply a gas to the distal end section body side and communicates with a gas feed source, and a nozzle having a jet outlet which jets a gas/liquid mixture fluid, in which the liquid supplied from the liquid feed path and the gas supplied from the gas feed path are mixed, toward the observation window, wherein an opening end of the liquid feed path and an opening end of the gas feed path are disposed in an order of proximity to the jet outlet.

### CROSS-REFERENCE TO RELATED APPLICATIONS

This application is based upon and claims the benefit of priority from prior Japanese Patent Application No. 2008-091918, filed Mar. 31, 2008, the entire contents of which are incorporated herein by reference.

### BACKGROUND OF THE INVENTION

1. Field of the Invention

The present invention relates to an endoscope including a fluid jet nozzle for cleaning, e.g. contamination adhering to an observation window which is provided on a distal end section body of the endoscope, a distal end cap-equipped endoscope, and an endoscope cleaning sheath.

2. Description of the Related Art

In a medical endoscope, an insertion section, which is inserted into a body cavity, is provided with an illumination window and an observation window at a distal end section body thereof. While emitting illumination light from the illumination window and illuminating the body cavity, observation is performed through the observation window. The distal end section body is provided with a fluid jet nozzle. In a case where blood, mucus or the like adheres to the illumination window or observation window and the field of vision is deteriorated, water and air are jetted from the fluid jet nozzle, thereby to clean the illumination window or observation window.

Jpn. Pat. Appln. KOKAI Publication No. H11-188004 (patent document 1) discloses the following structure. A distal end cap is detachably attached to a distal end section body of an insertion section of an endoscope. This distal end cap includes the air/water feed nozzle as mentioned above.

An air feed path and a water feed path are formed in the distal end section body. Distal end portions of a water feed tube and an air feed tube are connected, respectively, to the air feed path and the water feed path. A communication path, at which the air feed path and the water feed path are made confluent, is provided in the distal end section body. The air/water feed nozzle is connected to the communication path.

Proximal end portions of the water feed tube and air feed tube are connected to water feed means and air feed means on the proximal side of the insertion section. Water and air are supplied to the water feed tube and air feed tube from the water feed means and air feed means on the proximal side of the insertion section. In this structure, the water, which is fed from the water feed tube, and the air, which is fed from the air feed tube, are supplied to the air/water feed nozzle via the communication path in the distal end section body, and the water and air are jetted from the air/water feed nozzle to the observation window.

Jpn. Pat. Appln. KOKAI Publication No. H10-151108 (patent document 2), like patent document 1, discloses a structure wherein a water feed tube and an air feed tube are connected to a water feed path and an air feed path in a distal end section body of an endoscope, and the water feed tube and the air feed tube are made confluent in a communication path which is provided in the distal end section body. In addition, an air/water feed nozzle having a distal end portion with a reduced diameter is connected to the communication path. In this structure, water and air are jetted from the air/water feed nozzle to the observation window.

Jpn. Pat. Appln. KOKAI Publication No. H7-136102 (patent document 3) discloses the following structure. An air feed outlet and a water feed outlet, which open at a distal end face of a distal end potion body of an insertion section of an endoscope, are provided adjacent to each other. In the distal end section body, a nozzle is detachably attached in such a manner that the nozzle is opposed to the air feed outlet and water feed outlet. In this structure, the direction of jet of the air, which is fed from the air feed outlet, and the direction of jet of the water, which is fed from the water feed outlet, are varied by the nozzle, and the air and water are jetted toward the observation window.

Jpn. Pat. Appln. KOKAI Publication No. H6-14870 (patent document 4) discloses the following structure. An air feed path and a water feed path are provided in an insertion section of an endoscope. The air feed path and water feed path are made confluent in the insertion section, and made to communicate with an air/water feed nozzle. Further, air is intermittently blown into the water flowing in the water feed path, thus producing an air/water mixture fluid and enhancing the performance of cleaning.

### BRIEF SUMMARY OF THE INVENTION

According to a first aspect of the present invention, an endoscope comprising: an insertion section which is inserted in a body cavity; a distal end section body which constitutes a distal end section of the insertion section and has at least an observation window; a liquid feed path which is formed to supply a liquid to the distal end section body side and communicates with a liquid feed source; a gas feed path which is formed to supply a gas to the distal end section body side and communicates with a gas feed source; and a nozzle which is provided in the distal end section body and cleans the observation window by jetting toward the observation window a mixture fluid in which the liquid supplied from the liquid feed path and the gas supplied from the gas feed path are mixed, wherein the nozzle includes: a confluent portion which is provided in a mount plane in which the observation window of the distal end section body is provided, or in a plane parallel to the mount plane, the confluent portion making confluent and mixing the liquid supplied from the liquid feed path and the gas supplied from the gas feed path; and a jet outlet which jets a gas/liquid mixture fluid, which is mixed in the confluent portion, toward the observation window.

According to another aspect of the present invention, a distal end cap-equipped endoscope comprising: an insertion section which is inserted in a body cavity; a distal end section body which constitutes a distal end section of the insertion section and has at least an observation window; a liquid feed path which is formed to supply a liquid to the distal end section body side and communicates with a liquid feed source; a gas feed path which is formed to supply a gas to the distal end section body side and communicates with a gas feed source; a distal end cap which is detachably attached to the distal end section body; and a nozzle which is provided in the distal end cap and has a jet outlet which jets a mixture fluid, in which the liquid supplied from the liquid feed path and the gas supplied from the gas feed path are mixed, toward the observation window, wherein an opening end communicating with the liquid feed path and an opening end communicating with the gas feed path are successively disposed in an order of the liquid feed path and the gas feed path in an order of proximity to the jet outlet.

According to another aspect of the present invention, an endoscope cleaning sheath comprising: a cleaning sheath body which is fitted over an insertion section of an endoscope having at least an observation window at a distal end section body; a liquid feed path which is provided in the cleaning sheath body and communicates with a liquid feed source; a gas feed path which is provided in the cleaning sheath body and communicates with a gas feed source; and a nozzle which is provided in the cleaning sheath body and has a jet outlet which jets a gas/liquid mixture fluid, in which the liquid supplied from the liquid feed path and the gas supplied from the gas feed path are mixed, toward the observation window, wherein an opening end of the liquid feed path and an opening end of the gas feed path are successively disposed in an order of proximity to the jet outlet.

Advantages of the invention will be set forth in the description which follows, and in part will be obvious from the description, or may be learned by practice of the invention. Advantages of the invention may be realized and obtained by means of the instrumentalities and combinations particularly pointed out hereinafter.

### BRIEF DESCRIPTION OF THE SEVERAL VIEWS OF THE DRAWING

The accompanying drawings, which are incorporated in and constitute a part of the specification, illustrate embodiments of the invention, and together with the general description given above and the detailed description of the embodiments given below, serve to explain the principles of the invention.

FIG. 1A is a perspective view showing the entire structure of an endoscope and an endoscope cleaning sheath according to a first embodiment of the present invention;

FIG. 1B is a front view of a distal end section body of the endoscope according to the first embodiment;

FIG. 1C is a front view showing the relationship between a nozzle of the endoscope, an observation window and illumination windows in the first embodiment;

FIG. 2 is a transverse cross-sectional view of a sheath body of the endoscope according to the first embodiment;

FIG. 3 is a front view of a distal end section of the endoscope according to the first embodiment;

FIG. 4 is a cross-sectional view, taken along line IV-IV in FIG. 3, showing the endoscope according to the first embodiment;

FIG. 5 is a perspective view of a flexible endoscope according to a second embodiment of the present invention;

FIG. 6A is a perspective view showing a distal end section of the endoscope according to the second embodiment;

FIG. 6B is a cross-sectional view taken along line VIB-VIB in FIG. 6A;

FIG. 7A is a schematic front view of a nozzle according to a first modification of the second embodiment;

FIG. 7B is a schematic front view of a nozzle according to a second modification of the second embodiment;

FIG. 7C is a schematic front view of a nozzle according to a third modification of the second embodiment;

FIG. 7D is a schematic front view of a nozzle according to a fourth modification of the second embodiment;

FIG. 7E is a schematic front view of a nozzle according to a fifth modification of the second embodiment;

FIG. 7F is a schematic front view of a nozzle according to a sixth modification of the second embodiment;

FIG. 8 is a perspective view of a distal end cap-equipped endoscope according to a third embodiment of the invention; and

FIG. 9 is a longitudinal cross-sectional side view of a distal end section body of the distal end cap-equipped endoscope according to the third embodiment.

### DETAILED DESCRIPTION OF THE INVENTION

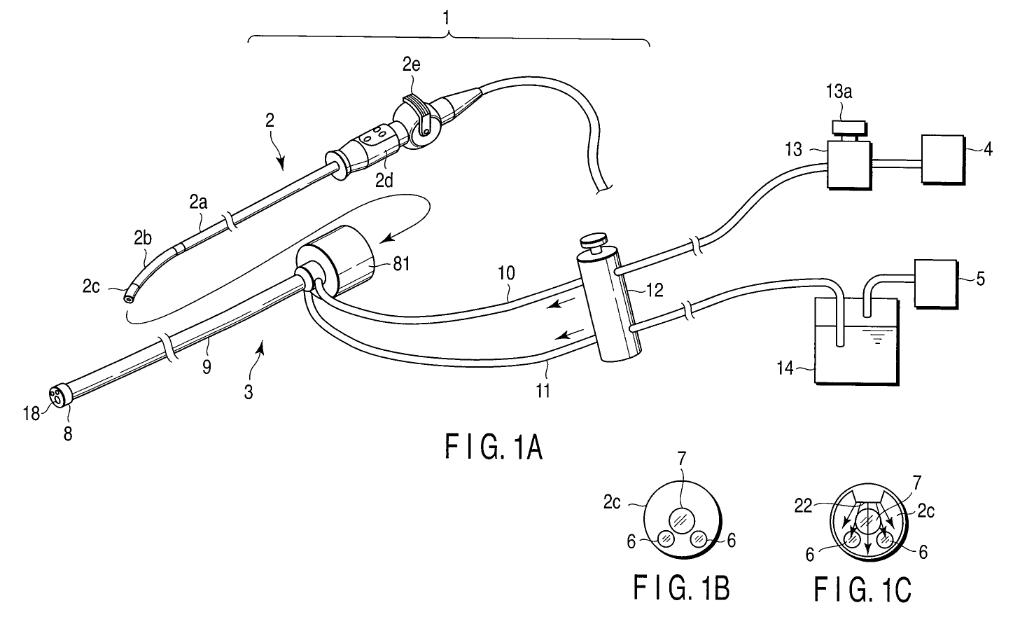

Embodiments of the present invention will now be described with reference to the accompanying drawings. FIG. 1A to FIG. 4 show a first embodiment of the present invention. FIG. 1A is a perspective view showing the entire structure of an endoscope apparatus 1, and FIG. 1B is a front view of a distal end section body 2c of an endoscope 2.

As shown in FIG. 1A, the endoscope apparatus 1 comprises the endoscope 2, an endoscope cleaning sheath 3, a gas feed pump 4 functioning as a gas feed device, and a liquid feed pump 5 functioning as a liquid feed device. The endoscope 2 is, for example, a rigid endoscope having a bending section 2b in an insertion section 2a thereof. A distal end portion of the insertion section 2a is provided with the distal end section body 2c. A proximal end portion of the insertion section 2a is provided with an operation section 2d. The operation section 2d is provided with a bending operation lever 2e for bending the bending section 2b in an up-and-down direction or in a right-and-left direction.

The endoscope cleaning sheath 3 is fitted over the insertion section 2a of the endoscope 2. Thereby, the endoscope cleaning sheath 3, as one piece with the insertion section 2a, is inserted into a body cavity. As shown in FIG. 1B, a distal end face of the distal end section body 2c is provided with two illumination windows 6 and one observation window 7. The illumination widows 6 constitute parts of an illumination optical system. The observation window 7 constitutes a part of an observation optical system.

The illumination window 6 is connected to a light source device (not shown) via a light guide fiber. The observation optical system is provided with an image pickup device including an image pickup element, such as a CCD, which photoelectrically converts an optical image, which is captured through the observation window 7, to an electric signal. A signal cable extends from the image pickup device. This signal cable is connected to an external camera control unit (not shown). Thus, reflective light from a subject, which is illuminated with illumination light that is emitted from the illumination window 6, is received as an optical image via the observation window 7. The optical image, after converted to the electric signal by the image pickup element, is transmitted to the camera control unit. The camera control unit generates a video signal on the basis of the electric signal, and outputs the video signal to, for example, a liquid crystal display which is a display device, thus displaying an endoscopic image on the screen of the liquid crystal display.

The endoscope cleaning sheath 3 is formed as an elongated cylindrical member. The insertion section 2a of the endoscope 2 is detachably inserted into the endoscope cleaning sheath 3. Thereby, the endoscope cleaning sheath 3 is disposed in a manner to cover the entirety of the insertion section 2a of the endoscope 2.

The endoscope cleaning sheath 3 is mainly composed of a distal end cover 8 which is a cylindrical body, and a tube body 9 which is composed of a multi-lumen tube. The distal end cover 8 is fitted on a distal end portion of the tube body 9. The proximal end side of the tube body 9 is provided with an operation section coupling unit 81 which has a greater diameter than the tube body 9. One end of a gas supply tube 10 and one end of a liquid supply tube 11 are coupled to the operation section coupling unit 81. The distal end cover 8 and the tube body may be integrally formed, or may be formed of the same material.

The other end of the gas supply tube 10 is connected to the gas feed pump (gas feed source) 4 via an opening/closing valve 12 and a pressure adjusting valve 13, which are provided at positions along the gas supply tube 10. The other end of the liquid supply tube 11 is connected to the liquid feed pump (liquid feed source) 5 via the opening/closing valve 12 and a liquid feed tank 14, which are provided at positions along the liquid supply tube 11.

The tube body 9, which is composed of the multi-lumen tube, is formed of a flexible material such as silicone, urethane or Teflon (trademark), or a rigid material such as polyamide, polyethylene, polypropylene or polycarbonate.

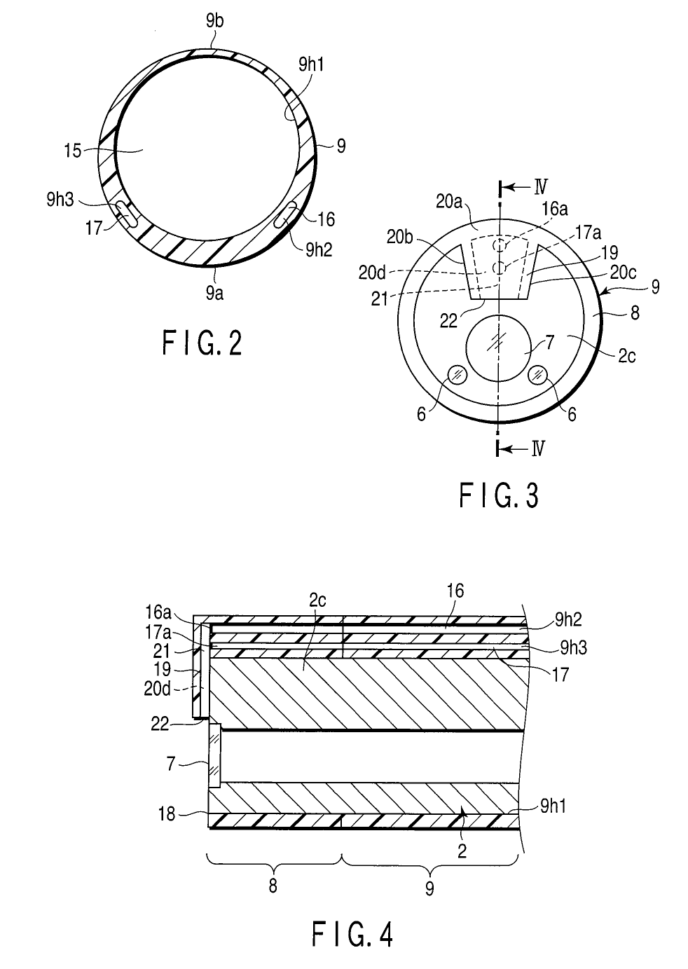

As shown in FIG. 2, the tube body 9 has an eccentric hole 9h1, the center axis of which is eccentric to the outer peripheral surface of the tube body 9. Thereby the peripheral wall of the tube body 9, which defines the eccentric hole 9h1, includes a large thickness portion 9a and a small thickness portion 9b. The eccentric hole 9h1 of the tube body 9 is a through-hole having openings at a distal end face and a proximal end face of the tube body 9. The eccentric hole 9h1 is used as an endoscope hole 15 in which the insertion section 2a of the endoscope 2 is inserted.

The large thickness portion 9a of the tube body 9 includes two through-holes 9h2 and 9h3, which penetrate the large thickness portion 9a in the axial direction of the tube body 9. One through-hole 9h2 is used as a gas feed path 16 serving as a first flow path for supplying a gas such as air. The other through-hole 9h3 is used as a liquid feed path 17 serving as a second flow path for supplying a liquid such as water or a cleaning solution. The proximal end side of the gas feed path 16 communicates with the gas supply tube 10, and the proximal end side of the liquid feed path 17 communicates with the liquid supply tube 11.

As shown in FIG. 4, the distal end cover 8 of the endoscope cleaning sheath 3 is a circular cylindrical member. The distal end cover 8 is provided with an opening portion 18 at a part thereof that is opposed to the front surface of the distal end section body 2c of the endoscope 2.

A nozzle 19, which is bent inward in a substantially L shape, is integrally provided in a front end portion of the distal end cover 8. As shown in FIG. 3, a space portion 20d is formed in the nozzle 19. The space portion 20d is surrounded by an outer peripheral wall 20a extending along the outer peripheral portion of the distal end section body 2c, and a left side wall 20b and a right side wall 20c, which define left and right side surfaces of the nozzle 19 in FIG. 3. The side walls 20b and 20c of the nozzle 19 may be formed in a tapering shape toward the observation window 7. The distal end cover 8, in which the nozzle 19 is integrally formed, may be formed of such rigid materials as polyamide, polyethylene, polypropylene and polycarbonate, thereby to maintain the shape of the nozzle.

Further, an opening end 16a, which communicates with the gas feed path 16, and an opening end 17a, which communicates with the liquid feed path 17, are open to the space portion 20d of the nozzle 19. The gas supplied from the gas feed path 16 and the liquid supplied from the liquid feed path 17 are made confluent within the space portion 20d of the nozzle 19. A confluent portion 21, which combines and mixes the gas and the liquid, is provided in the space portion 20d. A jet outlet 22, which is opposed to a side of the observation window 7 of the distal end section body 2c, is open at a distal end (a lower end portion in FIG. 3 and FIG. 4) of the nozzle 19.

In the nozzle 19, in the order of proximity to the jet outlet 22, the opening end 17a of the liquid feed path 17 and the opening end 16a of the gas feed path 16 are successively disposed. Thereby, the liquid, which is supplied from the opening end 17a of the liquid feed path 17, is pushed toward the jet outlet 22 by the gas which is supplied from the opening end 16a of the gas feed path 16 that is disposed on the rear side. In this manner, by disposing the opening end 17a of the liquid feed path 17 and the opening end 16a of the gas feed path 16 in the named order relative to the jet outlet 22, the liquid, which temporarily tries to stay, is pushed toward the jet outlet 22 by the jet pressure of the gas from the rear side. At this time, the gas that is supplied from the gas feed path 16 and the liquid that is supplied from the liquid feed path 17 are efficiently mixed into an atomized gas/liquid mixture fluid. This atomized gas/liquid mixture fluid is jetted toward the observation window 7 from the jet outlet 22.

In the present embodiment, the opening diameter of the opening end 16a of the gas feed path 16 is set to be equal to the opening diameter of the opening end 17a of the gas feed path 17. Alternatively, the opening diameter of the opening end 16a of the gas feed path 16 may be set to be greater than the opening diameter of the opening end 17a of the gas feed path 17. In this case, the flow amount of the gas becomes greater than the flow amount of the liquid. Thereby, such an advantage can be obtained that the gas and liquid are efficiently mixed when the gas and liquid collide with each other.

The confluent portion 21 of the nozzle 19 having the above-described structure is provided on a plane which continuously extends toward the observation window 7 that is provided on the distal end section body 2c. Thus, the gas supplied from the gas feed path 16 and the liquid supplied from the liquid feed path 17 are mixed in the confluent portion 21 into an atomized gas/liquid mixture fluid. This atomized gas/liquid mixture fluid is jetted from the jet outlet 22 toward the observation window 7 and illumination windows 6. Thereby, contamination (mucus, blood, etc.) adhering to the observation window 7 is blown off and cleaned by the atomized gas/liquid mixture fluid that is jetted from the jet outlet 22. In the present embodiment, since the illumination windows are also provided on the plane that is continuous with the confluent portion 21 of the nozzle 19, contamination adhering to the illumination windows can be blown off and cleaned at the same time.

Next, the operation of the first embodiment is described. When the endoscope 1 is used, the endoscope cleaning sheath 3 is set in advance in the state in which the endoscope cleaning sheath 3 is fitted over the insertion section 2a of the endoscope 2. At this time, the entirety of the insertion section 2a is covered with the tube body 9. The distal end section body 2c of the endoscope 2 is covered with the distal end cover 8. The jet outlet 22 of the nozzle 19 of the distal end cover 8 is disposed to be directed to the observation window 7 and illumination windows 6 of the distal end section body 2c.

In the state in which the endoscope cleaning sheath 3 is fitted over the endoscope 2, the insertion section 2a of the endoscope 2, as one piece with the endoscope cleaning sheath 3, is inserted into a body cavity of a patient. The inside of the body cavity is observed by the endoscope 2, and a diseased part is treated, where necessary. At this time, there is a case in which contamination adheres to the observation window 7 and the field of vision is deteriorated, or contamination adheres to the illumination window 6 and the illuminance lowers. In this case, the observation window 7 and illumination windows 6 can be cleaned remotely by the operation, which will be described below.

In the meantime, even in the case the illumination windows 6 are disposed on the side opposite to the nozzle 19 with respect to the observation window 7, the fluid is jetted from the jet outlet 22 in a spreading fashion, and thus the illumination windows 6 can also be cleaned.

Specifically, at the time of the work of cleaning the observation window 7 and illumination windows 6, the gas feed pump 4 is driven to feed gas and simultaneously the liquid feed pump 5 is driven to feed liquid. If the gas is fed from the gas feed pump 4, the gas is supplied to the gas feed path 16 via the gas supply tube 10. If the liquid is fed from the liquid feed pump 5, the liquid is supplied to the liquid feed path 17 via the liquid supply tube 11. Further, the gas in the gas feed path 16 and the liquid in the liquid feed path 17 are supplied into the nozzle 19. At this time, the gas supplied from the gas feed path 16 is fed in a manner to push out, from the rear side, the liquid that is supplied from the liquid feed path 17. Thereby, the gas and the liquid collide and are made turbulent in the confluent portion 21, and the liquid and gas are mixed into an atomized gas/liquid mixture fluid. The gas/liquid mixture fluid is jetted from the jet outlet 22 toward the observation window 7 and illumination windows 6.

At this time, since the confluent portion 21 of the nozzle 19 is provided on the plane that is continuous with the observation window 7 and illumination windows 6 of the distal end section body 2c, the atomized gas/liquid mixture fluid, which is mixed in the confluent portion 21, is jetted from the jet outlet 22 in the surface direction of the observation window 7 and illumination windows 6. As a result, the contamination adhering to the observation window 7 and illumination windows 6 can efficiently be blown off and cleaned by the gas/liquid mixture fluid that is jetted from the jet outlet 22. Moreover, since the gas/liquid mixture fluid is the atomized fluid, no drops of water remain on the surfaces of the observation window 7 and illumination windows 6, and the field of vision, as well as the illuminance, can instantaneously be secured.

In the case where the force of the atomized gas/liquid mixture fluid, which is jetted from the jet outlet 22, is to be increased, an adjustment knob 13a of the pressure adjusting valve 13 is controlled to increase the pressure of the gas. Thereby, the pressure of the gas that is supplied from the gas feed path 16 can be increased, and the gas/liquid mixture ratio can arbitrarily be controlled. In this case, in consideration of the difference in specific gravity between the gas and the liquid, the pressures of the gas feed pump 4 and liquid feed pump 5 may be preset to meet the relationship, i.e. the pressure of the gas feed pump 4>the pressure of the liquid feed pump 5.

The above-described first embodiment is directed to the case in which the endoscope cleaning sheath 3 is fitted over the rigid endoscope 2. Needless to say, however, the invention is applicable to the case in which the endoscope cleaning sheath 3 is fitted over a flexible endoscope.

According to the present invention, the following advantages can be obtained. Specifically, even with the simple structure in which the opening ends of the liquid feed path and gas feed path are disposed in the named order relative to the jet outlet, the liquid from the liquid feed path and the gas from the gas feed path are mixed, the atomized gas/liquid mixture fluid is splayed on the observation window, and the observation window can efficiently be cleaned.

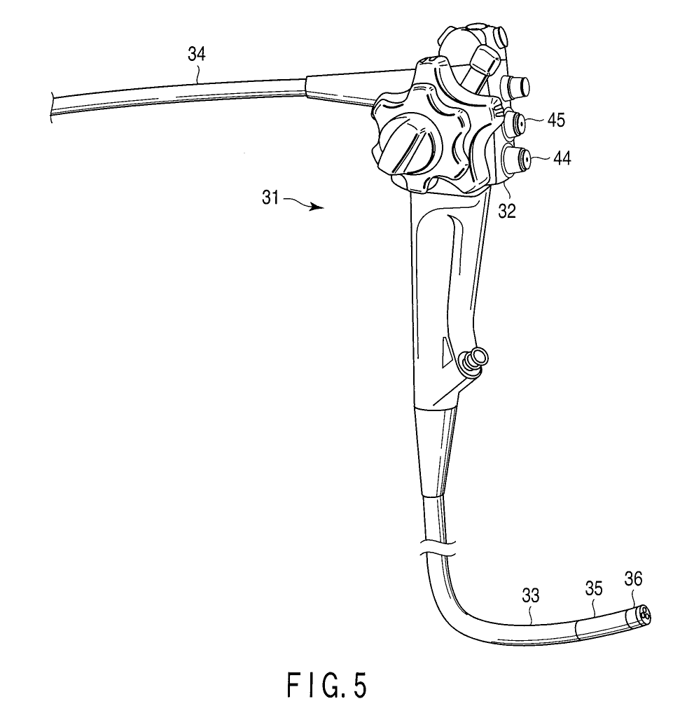

FIG. 5 to FIG. 7 show a second embodiment of the present invention. In this embodiment, a flexible endoscope is integrally equipped with a cleaning function. FIG. 5 is a perspective view showing the entirety of the flexible endoscope.

As shown in FIG. 5, in a flexible endoscope 31, one end of a flexible insertion section 33 and one end of a flexible universal cord 34 are coupled to an operation section 32. A distal end section body 36 is provided on the other end of the insertion section 33 via a bending section 35.

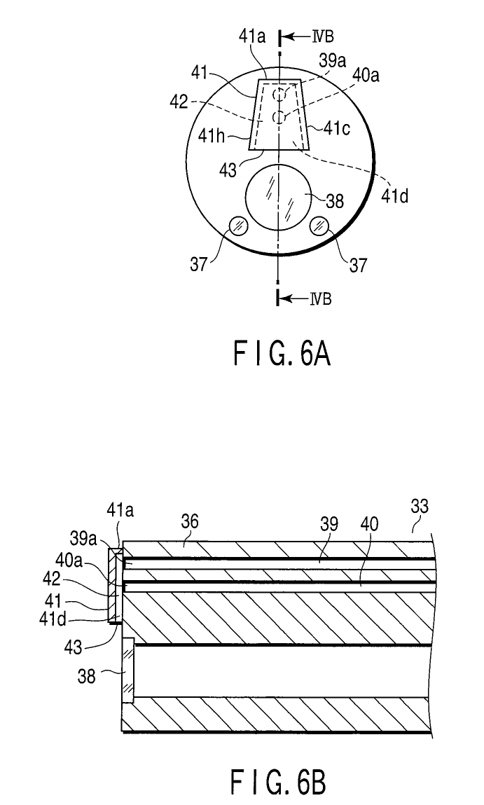

As shown in FIG. 6A, the distal end face of the distal end section body 36 is provided with one observation window 38 and two illumination windows 37. The observation window 38 constitutes a part of an observation optical system. The observation optical system is provided with an image pickup device including an image pickup element, such as a CCD, which photoelectrically converts an optical image, which is captured through the observation window 38, to an electric signal.

The insertion section 33 is provided with a gas feed path 39 for supplying a gas such as air, and a liquid feed path 40 for supplying a liquid such as water or a cleaning solution. Like the first embodiment, the gas feed path 39 and liquid feed path 40 communicate with the gas feed pump 4 and liquid feed pump 5 through the insertion section 33, operation section 32 and universal cord 34.

A nozzle 41 is integrally provided at a front end portion of the distal end section body 36 of the endoscope 31. The nozzle 41 has a space portion 41d which is surrounded by an upper wall 41a extending along the outer peripheral portion of the distal end section body 36, and left and right side walls (left side wall 41b and right side wall 41c) in FIG. 6A. As shown in FIG. 6B, an opening end 39a of the gas feed path 39 and an opening end 40a of the liquid feed path 40 are open to the space portion 41d of the nozzle 41. The gas supplied from the gas feed path 39 and the liquid supplied from the liquid feed path 40 are made confluent in the space portion 41d of the nozzle 41. A confluent portion 42, which combines and mixes the gas and the liquid, is provided in the space portion 41d. Further, a jet outlet 43 is open at a lower end portion (in FIG. 6A and FIG. 6B) of the nozzle 41. The jet outlet 43 is opposed to a side of the observation window 38 of the distal end section body 36.

In the nozzle 41, in the order of proximity to the jet outlet 43, the opening end 40a of the liquid feed path 40 and the opening end 39a of the gas feed path 39 are successively disposed. The liquid, which is supplied from the opening end 40a of the liquid feed path 40, is pushed toward the jet outlet 43 by the gas which is supplied from the opening end 39a of the gas feed path 39 that is disposed on the rear side. In this manner, by successively disposing the opening end 40a of the liquid feed path 40 and the opening end 39a of the gas feed path 39 relative to the jet outlet 43 in the order of proximity to the jet outlet 43, the liquid, which temporarily tries to stay in the confluent portion 42, is pushed toward the jet outlet 43 by the jet pressure of the gas from the rear side. Thus, the gas that is supplied from the gas feed path 39 and the liquid that is supplied from the liquid feed path 40 are efficiently mixed into an atomized gas/liquid mixture fluid. The atomized gas/liquid mixture fluid is jetted toward the observation window 38 from the jet outlet 43.

In the present embodiment, the opening diameter of the opening end 39a of the gas feed path 39 is set to be equal to the opening diameter of the opening end 40a of the gas feed path 40. Alternatively, the opening diameter of the opening end 39a of the gas feed path 39 may be set to be greater than the opening diameter of the opening end 40a of the gas feed path 40. In this case, the flow amount of the gas becomes greater than the flow amount of the liquid. Thereby, such an advantage can be obtained that the gas and liquid are efficiently mixed when the gas and liquid collide with each other.

The confluent portion 42 of the nozzle 41 having the above-described structure is provided on a plane which continuously extends toward the observation window 38 that is provided on the distal end section body 36. Thus, the gas supplied from the gas feed path 39 and the liquid supplied from the liquid feed path 40 collide in the confluent portion 21, and are made turbulent and mixed into an atomized gas/liquid mixture fluid. This atomized gas/liquid mixture fluid is jetted from the jet outlet 43 toward the observation window 38. Thereby, the contamination adhering to the observation window 38 is blown off and cleaned by the atomized gas/liquid mixture fluid that is jetted from the jet outlet 43.

Further, the operation section 32 is provided with a gas/liquid feed button 44 and a suction button 45. The gas/liquid feed button 44 controls the flow amount of the gas supplied from the gas feed path 39 and the flow amount of the liquid supplied from the liquid feed path 40, and controls the gas/liquid mixture fluid that is jetted in an atomized state from the jet outlet 43 of the nozzle 41. Although not shown, like the first embodiment, the nozzle, observation window and illumination windows may be arranged in the named order, so that the observation window and illumination windows of the nozzle may be cleaned.

Next, the operation of the second embodiment is described. In this embodiment, the nozzle 41 is integrally provided on the distal end section body 36 of the insertion section 33 of the endoscope 31. The jet outlet 43 of the nozzle 41 is opposed to the side of the observation window 38. The insertion section 33 of the endoscope 31 is inserted into a body cavity of a patient, and the inside of the body cavity is observed and a diseased part is treated, where necessary. At this time, if contamination adheres to the observation window 38 and the field of vision is deteriorated, the observation window 38 can be cleaned remotely by the operation described below.

Specifically, at the time of the work of cleaning the observation window 38, the gas feed pump 4 is driven to feed gas and simultaneously the liquid feed pump 5 is driven to feed liquid. If the gas is fed from the gas feed pump 4, the gas is supplied to the gas feed path 39. If the liquid is fed from the liquid feed pump 5, the liquid is supplied to the liquid feed path 40. Then, the gas in the gas feed path 39 and the liquid in the liquid feed path 40 are supplied into the nozzle 41. At this time, the gas supplied from the gas feed path 39 and the liquid supplied from the liquid feed path 40 are mixed in the confluent portion 42 into an atomized gas/liquid mixture fluid. The atomized gas/liquid mixture fluid is jetted from the jet outlet 43 toward the observation window 38 and illumination windows 37.

At this time, the gas in the gas feed path 39 and the liquid in the liquid feed path 40 are supplied into the nozzle 41. The gas supplied from the gas feed path 39 pushes out, from the rear side, the liquid that is supplied from the liquid feed path 40. Thereby, the gas and the liquid collide and are made turbulent in the confluent portion 42, and the liquid and gas are mixed into an atomized gas/liquid mixture fluid. The gas/liquid mixture fluid is jetted from the jet outlet 43 toward the observation window 38.

Moreover, since the confluent portion 42 of the nozzle 41 is provided on the plane that is continuous with the observation window 38 of the distal end section body 36, the atomized gas/liquid mixture fluid, which is mixed in the confluent portion 42, is jetted from the jet outlet 43 to the observation window 38. As a result, the contamination adhering to the observation window 38 can efficiently be blown off and cleaned by the gas/liquid mixture fluid that is jetted from the jet outlet 43. Furthermore, since the gas/liquid mixture fluid is the atomized fluid, particles of water are fine and immediately evaporate. Thus, no drops of water remain on the surface of the observation window 38, and the field of vision, as well as the illuminance, can instantaneously be secured.

In the case where the force of the atomized gas/liquid mixture fluid, which is jetted from the jet outlet 43, is to be increased, the gas/liquid feed button 44 of the operation section 32 is controlled to increase the pressure of the gas. Thereby, the pressure of the gas that is supplied from the gas feed path 39 can be increased, and the gas/liquid mixture ratio can arbitrarily be controlled.

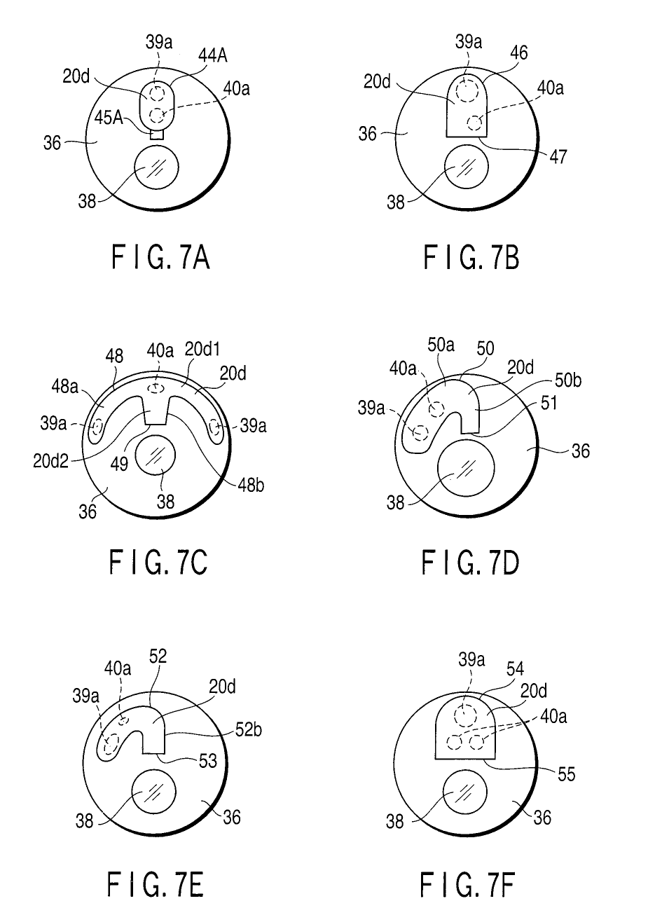

FIG. 7A to FIG. 7F show different modifications of the nozzle 41 of the second embodiment. Each of FIG. 7A to FIG. 7F is a schematic front view of the distal end section body 36.

FIG. 7A shows a nozzle 44A according to a first modification of the nozzle 41 of the second embodiment. The nozzle 44A shown in FIG. 7A is formed in an elliptic shape on the front end face of the distal end section body 36. An elliptic space portion 20d is provided within the nozzle 44A.

A jet outlet 45A, which projects and opens toward the observation window 38, is provided on a central portion side of the distal end section body 36. An opening end 40a of the liquid feed path 40 and an opening end 39a of the gas feed path 39 are provided in the nozzle 44A. The opening end 40a of the liquid feed path 40 and the opening end 39a of the gas feed path 39 are arranged in the order of proximity to the jet outlet 45A along the longitudinal axis of the elliptic space portion 20d.

The liquid, which is supplied from the opening end 40a of the liquid feed path 40, is supplied, while being pushed toward the jet outlet 45A by the gas which is supplied from the opening end 39a of the gas feed path 39 that is disposed on the rear side.

FIG. 7B shows a nozzle 46 according to a second modification of the nozzle 41 of the second embodiment. The nozzle 46 shown in FIG. 7B is formed in an elongated shape on the front end face of the distal end section body 36. An elongated space portion 20d is provided within the nozzle 46. The nozzle 46 is provided with a jet outlet 47 at an end portion thereof on a central portion side of the distal end section body 36. The jet outlet 47 is open to the observation window 38.

An opening end 40a of the liquid feed path 40 and an opening end 39a of the gas feed path 39 are provided in the nozzle 46. The opening end 40a of the liquid feed path 40 is disposed in the vicinity of the jet outlet 47. The opening end 39a of the gas feed path 39 is disposed at a position farther from the jet outlet 47 than the opening end 40a of the liquid feed path 40.

The liquid, which is supplied from the opening end 40a of the liquid feed path 40, is supplied, while being pushed toward the jet outlet 47 by the gas which is supplied from the opening end 39a of the gas feed path 39 that is disposed more on the rear side than the opening end 40a.

In this modification, the opening diameter of the opening end 39a of the gas feed path 39 may be set to be greater than the opening diameter of the opening end 40a of the gas feed path 40. Accordingly, the flow amount of the gas that is supplied from the gas feed path 39 is greater than the flow amount of the liquid that is supplied from the liquid feed path 40, and the efficiency in mixture of the gas and liquid is further enhanced. In addition, a turbulent flow easily occurs by disposing the opening ends 39a and 40a with a displacement from the longitudinal axis of the space portion 20d.

FIG. 7C shows a nozzle 48 according to a third modification of the nozzle 41 of the second embodiment. The nozzle 48 shown in FIG. 7C includes an arcuate portion 48a with a curvature according to the outer peripheral portion of the distal end section body 36, and a projection portion 48b which projects from an intermediate part of the arcuate portion 48a toward the central part of the distal end section body 36. Similarly, a space portion 20d within the nozzle 48 includes an arcuate portion 20d1 and a projection portion 20d2.

A jet outlet 49, which projects and opens toward the observation window 38, is provided at the projection portion 48b of the nozzle 48. Opening ends 39a of the gas feed path 39 are provided at both end portions of the arcuate portion 48a of the nozzle 48. An opening end 40a of the liquid feed path 40 is provided at a central part of the nozzle 48. Thus, the opening end 40a of the liquid feed path 40 and the opening ends 39a of the gas feed path 39 are successively disposed in the nozzle 48 in the order of proximity to the jet outlet 49.

The liquid that is supplied from the opening end 40a of the liquid feed path 40 is mixed with the gas that is supplied from the opening ends 39a of the gas feed path 39, which are disposed on the rear side. The liquid is supplied, while being pushed toward the jet outlet 49 by the pressure of the gas.

In the present embodiment, the opening diameter of each of the opening ends 39a of the gas feed path 39 is set to be greater than the opening diameter of the opening end 40a of the liquid feed path 40. In this case, the liquid and gas are easily mixed since the liquid from the opening end 40a is sandwiched by the gas from the two opening ends 39a.

FIG. 7D shows a nozzle 50 according to a fourth modification of the nozzle 41 of the second embodiment. The nozzle 50 shown in FIG. 7D is formed in an inverted U shape including an arcuate portion 50a and a projection portion 50b. The arcuate portion 50a is formed in an arcuate shape with a curvature according to the outer peripheral portion of the distal end section body 36. The projection portion 48b is formed in a shape projecting from one end of the arcuate portion 50a toward the central part of the distal end section body 36.

A jet outlet 51, which is open toward a side portion of the observation window 38 that is disposed on the central portion side of the distal end section body 36, is provided at the distal end side of the projection portion 50b of the nozzle 50. An opening end 40a of the liquid feed path 40 and an opening end 39a of the gas feed path 39 are provided within the arcuate portion 50a of the nozzle 50. The opening end 40a of the liquid feed path 40 and the opening end 39a of the gas feed path 39 are successively disposed in the order of proximity to the jet outlet 51. Thereby, the liquid that is supplied from the opening end 40a of the liquid feed path 40 is supplied, while being pushed toward the jet outlet 51 by the gas that is supplied from the opening end 39a of the gas feed path 39 which is disposed on the rear side.

The arcuate portion 50a of the space portion 20d of the nozzle 50 has a shape with a curvature. Accordingly, since the flow speed differs between the inner peripheral area and the outer peripheral area of the arcuate portion 50a, a turbulent flow easily occurs and a gas/liquid mixture fluid can easily be produced.

FIG. 7E shows a nozzle 52 according to a fifth modification of the nozzle 41 of the second embodiment. The nozzle 52 shown in FIG. 7E is formed in an inverted U shape including an arcuate portion 52a and a projection portion 52b. The arcuate portion 52a is formed in an arcuate shape with a curvature according to the outer peripheral portion of the distal end section body 36. The projection portion 52b is formed in a shape projecting from one end of the arcuate portion 52a toward the central part of the distal end section body 36.

A jet outlet 53, which is open toward a side portion of the observation window 38 that is disposed on the central portion side of the distal end section body 36, is provided at the distal end side of the projection portion 52b of the nozzle 52. An opening end 40a of the liquid feed path 40 and an opening end 39a of the gas feed path 39 are provided within the arcuate portion 52a of the nozzle 52. The opening end 40a of the liquid feed path 40 and the opening end 39a of the gas feed path 39 are successively disposed in the order of proximity to the jet outlet 53. Thereby, the liquid that is supplied from the opening end 40a of the liquid feed path 40 is supplied, while being pushed toward the jet outlet 53 by the gas that is supplied from the opening end 39a of the gas feed path 39 which is disposed on the rear side.

In the present embodiment, the opening diameter of the opening end 39a of the gas feed path 39 is set to be greater than the opening diameter of the opening end 40a of the liquid feed path 40.

The arcuate portion 52a of the space portion 20d of the nozzle 52 has a shape with a curvature. Accordingly, since the flow speed differs between the inner peripheral area and the outer peripheral area of the arcuate portion 52a, a turbulent flow easily occurs and a gas/liquid mixture fluid can easily be produced.

FIG. 7F shows a nozzle 54 according to a sixth modification of the nozzle 41 of the second embodiment. The nozzle 54 shown in FIG. 7F is formed in a semi-elliptic shape on the front end face of the distal end section body 36. A semi-elliptic space portion 20d is provided within the nozzle 54.

The nozzle 54 is provided with a jet outlet 55 at an end portion thereof on a central portion side of the distal end section body 36. The jet outlet 55 is open toward a side portion of the observation window 38.

Opening ends 40a of two liquid feed paths 40 and an opening end 39a of one gas feed path 39 are provided in the nozzle 54. The opening ends 40a of the two liquid feed paths 40 are disposed in the vicinity of the jet outlet 55. The opening end 39a of the gas feed path 39 is disposed at a position farther from the jet outlet 55 than the opening ends 40a of the liquid feed paths 40.

The liquid, which is supplied from the opening ends 40a of the liquid feed paths 40, is supplied, while being pushed toward the jet outlet 55 by the gas which is supplied from the opening end 39a of the gas feed path 39 that is disposed on the rear side.

In this modification, the diameter of each of the opening ends 40a of the gas feed paths 40 is set to be smaller than the diameter of the opening end 39a of the gas feed path 39. Accordingly, water drops with a small flow amount can easily be formed, and spraying with high cleaning performance can be performed by blowing such water drops.

In each of the above-described modifications, the confluent portion of the nozzle 44A, 46, 48, 50, 52, 54, is provided on the plane that is continuous with the observation window 38 of the distal end section body 36. Therefore, the atomized gas/liquid mixture fluid, which is mixed in the confluent portion, is jetted toward the observation window 38, and the contamination adhering to the observation window 38 can efficiently be blown off and cleaned. Moreover, since the gas/liquid mixture fluid is the atomized fluid, no drops of water remain on the surface of the observation window 38, and the field of vision can instantaneously be secured.

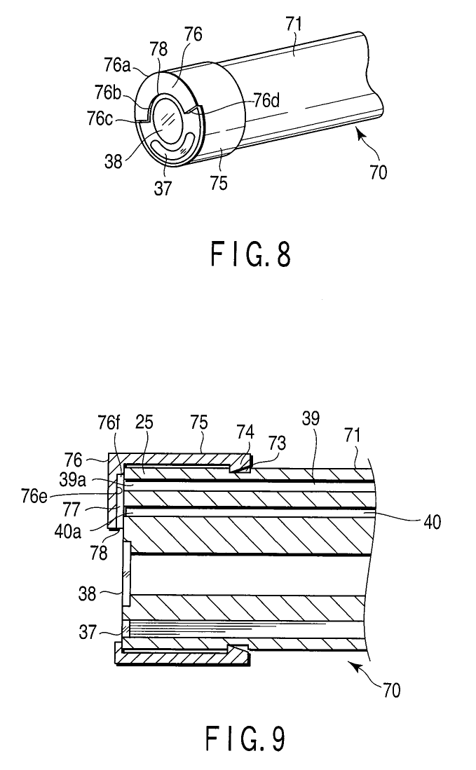

FIG. 8 and FIG. 9 show a third embodiment of the invention. The structural parts common to those in the second embodiment (see FIG. 5 to FIG. 7) are denoted by like reference numerals, and a description thereof is omitted.

The present embodiment shows a distal end cap-equipped endoscope. As shown in FIG. 8, an observation window 38 and an illumination window 37 are provided on a distal end section body 72 of an insertion section 71 of a flexible endoscope 70. In addition, as shown in FIG. 9, a gas feed path 39 and a liquid feed path 40 are provided in the insertion section 71. An opening end 39a communicating with the gas feed path 39 and an opening end 40a communicating with the liquid feed path 40 are provided at a front end face of the distal end section body 72.

Further, an annular engagement groove 73 is provided on an outer peripheral surface of the distal end section body 72. A circular cylindrical distal end cap 75 is detachably engaged with the distal end section body 72. The distal end cap 75 has, at its rear end portion, an engagement projection 74 which is engaged with the engagement groove 73. A front end portion of the distal end cap 75 is integrally provided with an arcuate nozzle 76 along the outer peripheral portion of the distal end cap 75. The nozzle 76 includes a space portion 76f which is surrounded by an outer peripheral wall 76a extending along the outer peripheral portion of the distal end cap 75, an inner peripheral wall 76b surrounding a part of the outer periphery of the observation window 38, left and right end walls 76c and 76d which define left and right side surfaces of the nozzle 76 in FIG. 8, and an arcuate front wall 76e. The space portion 76f is curved in an arcuate shape according to the curvature of the outer peripheral wall 76a and inner peripheral wall 76b.

The gas feed path 39 and liquid feed path 40 are open toward the front end side of the distal end cap 75 within the space portion 76f of the nozzle 76. The opening end 39a of the gas feed path 39 and the opening end 40a of the liquid feed path 40 are opposed to the inner surface of the front wall 76e of the nozzle 76.

An intermediate part in the longitudinal direction of the nozzle 76 is provided with a confluent portion 77. The confluent portion 77 combines and mixes the gas that is supplied from the gas feed path 39 and the liquid that is supplied from the liquid feed path 40. A jet outlet 78 is provided in the inner peripheral wall 76b of the nozzle 76, which is opposed to the confluent portion 77. The jet outlet 78 is configured to jet the gas/liquid mixture fluid, which is made confluent and mixed in the confluent portion 77, toward the observation window 38.

In the nozzle 19, in the order of proximity to the jet outlet 78, the opening end 40a of the liquid feed path 40 and the opening end 39a of the gas feed path 39 are successively disposed. Thereby, the liquid, which is supplied from the opening end 40a of the liquid feed path 40, is pushed toward the jet outlet 78, even if the liquid temporarily tries to stay in the nozzle 76, by the jet pressure of the gas which is supplied from the opening end 39a of the gas feed path 39 that is disposed on the rear side. At this time, the gas that is supplied from the gas feed path 39 and the liquid that is supplied from the liquid feed path 40 are efficiently mixed into an atomized gas/liquid mixture fluid. This atomized gas/liquid mixture fluid is jetted toward the observation window 38 from the jet outlet 78.

The confluent portion 77 of the nozzle 76 having the above-described structure is provided on the same plane as the observation window 38 of the distal end section body 72. Thereby, the gas supplied from the air feed path 39 and the liquid supplied from the liquid feed path 40 are mixed in the confluent portion 77 into an atomized gas/liquid mixture fluid. The atomized gas/liquid mixture fluid is jetted from the jet outlet 78 toward the observation window 38. Therefore, the contamination adhering to the observation window 38 can be blown off and cleaned.

Like the preceding embodiments, as shown in FIG. 8, the illumination window 37 is disposed on the plane that is continuous with the confluent portion 77 of the nozzle 76. Thereby, the illumination window 37 can similarly be cleaned. The present invention may be applied to only the illumination window. In this case, high luminance can be secured, and the efficiency of inspection can be enhanced.

The present invention is not limited directly to the above-described embodiments. In practice, the structural elements can be modified and embodied without departing from the spirit of the invention. Various inventions can be made by properly combining the structural elements disclosed in the embodiments. For example, some structural elements may be omitted from all the structural elements disclosed in the embodiments. Furthermore, structural elements in different embodiments may properly be combined.

Additional advantages and modifications will readily occur to those skilled in the art. Therefore, the invention in its broader aspects is not limited to the specific details and representative embodiments shown and described herein. Accordingly, various modifications may be made without departing from the spirit or scope of the general inventive concept as defined by the appended claims and their equivalents.

What is claimed is:

1. An endoscope comprising:
an insertion section which is inserted in a body cavity;
a distal end section body which constitutes a distal end section of the insertion section and has at least an observation window;
a liquid feed path which is formed to supply a liquid to the distal end section body side and communicates with a liquid feed source;
a gas feed path which is formed to supply a gas to the distal end section body side and communicates with a gas feed source; and
a nozzle having a jet outlet which jets a gas/liquid mixture fluid, in which the liquid supplied from the liquid feed path and the gas supplied from the gas feed path are mixed, toward the observation window,
wherein an opening end of the liquid feed path and an opening end of the gas feed path are disposed in an order of proximity to the jet outlet.

2. The endoscope according to claim 1, further comprising gas feed pressure control means for controlling a gas feed pressure for feeding the gas to the nozzle via the gas feed path.

3. The endoscope according to claim 1, wherein the nozzle includes a confluent portion which combines and mixes the gas supplied from the gas feed path with the liquid supplied from the liquid feed path, the confluent portion being provided on a side opposite to the observation window with respect to the jet outlet.

4. The endoscope according to claim 1, wherein an opening diameter of the opening end of the liquid feed path is smaller than an opening diameter of the opening end of the gas feed path.

5. The endoscope according to claim 1, wherein a plurality of the opening ends of the gas feed path and a plurality of the opening ends of the liquid feed path are provided in the nozzle.

6. The endoscope according to claim 1, wherein the opening end of the liquid feed path is provided at a confluent portion of the nozzle.

7. The endoscope according to claim 1, wherein a flow amount of the gas supplied from the gas feed path to the nozzle and a flow amount of the liquid supplied from the liquid feed path to the nozzle have a relationship: the flow amount of the gas>the flow amount of the liquid.

8. A distal end cap-equipped endoscope comprising:
an insertion section which is inserted in a body cavity;
a distal end section body which constitutes a distal end section of the insertion section and has at least an observation window;
a liquid feed path which is formed to supply a liquid to the distal end section body side and communicates with a liquid feed source;
a gas feed path which is formed to supply a gas to the distal end section body side and communicates with a gas feed source;
a distal end cap which is detachably attached to the distal end section body; and
a nozzle which is provided in the distal end cap and has a jet outlet which jets a mixture fluid, in which the liquid supplied from the liquid feed path and the gas supplied from the gas feed path are mixed, toward the observation window,
wherein an opening end communicating with the liquid feed path and an opening end communicating with the gas feed path are successively disposed in an order of the liquid feed path and the gas feed path in an order of proximity to the jet outlet.

9. The distal end cap-equipped endoscope according to claim 8, further comprising gas feed pressure control means for controlling a gas feed pressure for feeding the gas to the nozzle via the gas feed path.

10. The distal end cap-equipped endoscope according to claim 8, wherein the nozzle includes a confluent portion which combines and mixes the gas supplied from the gas feed path with the liquid supplied from the liquid feed path, the confluent portion being provided on a side opposite to the observation window with respect to the jet outlet.

11. The distal end cap-equipped endoscope according to claim 8, wherein an opening diameter of the opening end of the liquid feed path is smaller than an opening diameter of the opening end of the gas feed path.

12. The distal end cap-equipped endoscope according to claim 8, wherein a plurality of the opening ends of the gas feed path and a plurality of the opening ends of the liquid feed path are provided in the nozzle.

13. The distal end cap-equipped endoscope according to claim 8, wherein the opening end of the liquid feed path is provided at a confluent portion of the nozzle.

14. The distal end cap-equipped endoscope according to claim 8, wherein a flow amount of the gas supplied from the gas feed path to the nozzle and a flow amount of the liquid supplied from the liquid feed path to the nozzle have a relationship: the flow amount of the gas>the flow amount of the liquid.

15. An endoscope cleaning sheath comprising:
a cleaning sheath body which is fitted over an insertion section of an endoscope having at least an observation window at a distal end section body;
a liquid feed path which is provided in the cleaning sheath body and communicates with a liquid feed source;
a gas feed path which is provided in the cleaning sheath body and communicates with a gas feed source; and
a nozzle which is provided in the cleaning sheath body and has a jet outlet which jets a gas/liquid mixture fluid, in which the liquid supplied from the liquid feed path and the gas supplied from the gas feed path are mixed, toward the observation window,
wherein an opening end of the liquid feed path and an opening end of the gas feed path are successively disposed in an order of proximity to the jet outlet.

16. The endoscope cleaning sheath according to claim 15, further comprising gas feed pressure control means for controlling a gas feed pressure for feeding the gas to the nozzle via the gas feed path.

17. The endoscope cleaning sheath according to claim 15, wherein the nozzle includes a confluent portion which combines and mixes the gas supplied from the gas feed path with the liquid supplied from the liquid feed path, the confluent portion being provided on a side opposite to the observation window with respect to the jet outlet.

18. The endoscope cleaning sheath according to claim 15, wherein an opening diameter of the opening end of the liquid feed path is smaller than an opening diameter of the opening end of the gas feed path.

19. The endoscope cleaning sheath according to claim 15, wherein a plurality of the opening ends of the gas feed path and a plurality of the opening ends of the liquid feed path are provided in the nozzle.

20. The endoscope cleaning sheath according to claim 15, wherein the opening end of the liquid feed path is provided at a confluent portion of the nozzle.

21. The endoscope cleaning sheath according to claim 15, wherein a flow amount of the gas supplied from the gas feed path to the nozzle and a flow amount of the liquid supplied from the liquid feed path to the nozzle have a relationship: the flow amount of the gas>the flow amount of the liquid.
